(19) United States
(12) Patent Application Publication
(10) Pub. No.: US 2024/0245291 A1
(43) Pub. Date: Jul. 25, 2024

(54) AN INTELLIGENT SINGLE TOOL FOR CLINICIANS

(71) Applicant: NALIGANTI CORNEL SANTOSH KUMAR, Telangana (IN)

(72) Inventor: NALIGANTI CORNEL SANTOSH KUMAR, Telangana (IN)

(21) Appl. No.: 18/015,725

(22) PCT Filed: Dec. 8, 2021
(86) PCT No.: PCT/IB2021/061452

§ 371 Date: Jan. 12, 2023

(30) Foreign Application Priority Data
Aug. 6, 2021 (IN) 202141035573

(51) Int. Cl. A61B 3/00; A61B 3/12; A61B 3/14; A61B 5/00; G06F 3/01; G06T 7/00; G16H 10/60; G16H 40/67

(52) U.S. Cl. CPC A61B 3/0025; A61B 3/12; A61B 3/14; A61B 5/0013; A61B 5/0022; G06F 3/017; G06T 7/0012; G16H 10/60; G16H 40/67; A61B 2560/0431; G06T 2207/30041

(57) ABSTRACT

The present invention discloses a system for an Intelligent Single Tool for Clinicians for detecting abnormalities in eyes, brain, heart and kidney of a patient. The method comprising a sequential procedure for capturing and processing the retinal image by an edge device, which has an IOT sensor embedded in it, and connected to a FOG device, which simply relays the image captured by edge device to the cloud. Processing on the retinal image take place at the FOG device, which can be a mobile or a desktop/laptop or a server that configured with the AI application that interfaces with edge device on one side and with cloud on the other end and sending the captured, processed, and evaluated retinal image to a cloud server for extensive processing. Further, the processing of the retinal image facilitates determining 6 eye related diseases, and deriving abnormalities at the heart, brain and kidneys.

### TECHNICAL FIELD

The present invention relates to an IoT Based Detection and Derivation of Diseases and Tracing abnormalities in Eyes, Brain, Heart, and Kidney.

### BACKGROUND OF THE INVENTION

Imaging of human organs plays a critical role in diagnosis of multiple diseases. This is especially true for the human retina, where the presence of a large network of blood vessels and nerves make it a near-ideal window for exploring the effects of diseases that harm vision (such as diabetic retinopathy seen in diabetic patients, cytomegalovirus retinitis seen in HIV/ADS patients, glaucoma, and so forth) or other systemic diseases (such as hypertension, stroke, and so forth). Advances in computer-aided image processing and analysis technologies are essential to make imaging-based disease diagnosis scalable, cost-effective, and reproducible. Such advances would directly result in effective triage of patients, leading to timely treatment and better quality of life.

Ocular trauma is a significant cause of preventable visual impairment. Ocular injuries can account for up to a third of the casualties sustained by workers in hazardous or disaster environments; while untold others can experience other less devastating eye issues while on the job. Because the diagnosis and treatment of ocular trauma and disease are daunting to most non-ophthalmic providers, most op to refer ocular patients to local medics, ophthalmologists, or optometrists for evaluation or all but the most routine conditions.

Most clinicians, especially neuro-ophthalmologists and glaucoma specialists have been trying to understand whether the information yielded by optical coherence tomography (OCT) is really helping them to improve upon the clinical care of their patients. Since its inception. OCT had provided new information about the status of the optic nerve, in terms of axon loss, providing information about the thickness of the retinal nerve fiber layer (RNFL). As neuro-ophthalmologists have attempted to incorporate the quantification of RNFL thickness into their decision-making, they have gained new insight into both its clinical usefulness and its limitations. Recently, the use of the macula OCT has helped to further expand the usefulness of OCT in neuro-ophthalmology patients, especially as spectral domain OCT (SD-OCT) has improved resolution and shortened the time for sampling of tissue volume. More advanced image analysis algorithms have improved the ability to segment the retinal layers in the macula, allowing improved detection and differentiation of the cause of visual loss.

In our nomenclature, the ‘passive’ techniques refer to the standard way of acquiring ophthalmic images in which an operator takes an image, which is subsequently subjected to various image enhancement algorithms either before being analyzed by clinician or graded automatically by an algorithm. In ‘active’ image acquisition, multiple frames of the same structure are obtained either with automatic reconstruction or with interactive operator-assisted reconstruction of the image. In this present invention, focus on the ‘active’ paradigm, where clinically meaningful images would be reconstructed automatically from multiple acquisitions with varying image quality for An Intelligent Single Tool for Clinicians

### OBJECT OF THE INVENTION

The primary object of the present invention is to develop a system for an Intelligent Single Tool for Clinicians for detecting abnormalities related to Opthalmology, Cardiology, Neurology, and Nephrology of a patient.

Another object of the present invention is to provide a reliable method for an Intelligent Single Tool for Clinicians for detecting abnormalities related to Opthalmology, Cardiology, Neurology, and Nephrology of a patient.

Another object of the present invention is to provide a cost effective and instantaneous for an Intelligent Single Tool for Clinicians for detecting abnormalities related to Opthalmology. Cardiology. Neurology, and Nephrology of a patient.

These and other objects and advantages of the present invention will become readily apparent from the following detailed description taken in conjunction with the accompanying drawings.

### SUMMARY OF THE INVENTION

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

The various embodiments of the present invention provide a system for an Intelligent Single Tool for Clinicians for detecting abnormalities in Opthalmology. Cardiology, Neurology, and Nephrology of a patient, the system comprises an smart device with an IOT sensor and an external server, wherein the IOT sensor is an edge device with a communication unit, wherein the IOT sensor capture a retinal image of the patient to process the image, wherein to determine one or more eye related diseases; and a plurality of other diseases related to one or more of cardio-vascular, renal failure, nephropathy, and kidney.

According to an embodiment of the present invention the system further comprising a communication unit to receive electronic health records (EHR) via a FHIR (Fast Healthcare Interoperability Resources) API (application programming interface).

According to an embodiment of the present invention for diagnosis the communication unit to send data related to the processed mage to a cloud server for analyzing long-term patterns of the patient, when processing greater than processing capability of edge device is required.

According to an embodiment of the present invention the IOT sensor is an inertial measurement unit (IMU)-based actigraph configured to send data real time to a fog device that contains higher computing power that an edge device for gesture recognition.

According to an embodiment of the present invention the IOT sensor to clean data related to the retinal image and facilitate the fog node to handle sensor fusion of clinical one-dimensional (1D) biosignal.

According to an embodiment of the present invention the system, wherein a fog device is used to process the image, when the processing required is higher than the processing capability of the edge device.

According to an embodiment of the present invention, the system for an Intelligent Single Tool for Clinicians for detecting abnormalities in eyes, brain, heart and kidney of a patient. The method comprising a sequential procedure for capturing and processing the retinal image by an edge device, which has an IOT sensor embedded in it, and connected to a FOG device, which simply relays the image captured by edge device to the cloud. Here, the intelligent edge device with Artificial Intelligence has the capability to determine the kind of pathologies present in such patient under consideration, in line with additional processing that would be done at FOG device, if necessary. After detection of patient disease, the current patient record gets persistently stored at the cloud, wherein this data record used by patient (to know the progression or healing of the disease), researchers (to use the datasets for the development of novel and useful techniques to bring sophisticated solutions to ease the diagnostic procedures), when the workloads are needed to be resolved at FOG device(s) which is other side connected to the cloud.

According to an embodiment of the present invention, wherein any processing on the retinal image take place at the FOG device, wherein the FOG device can be a mobile or a desktop/laptop or a server/servers that is/are configured with the AI application that interfaces with edge device on one side and with cloud on the other end and sending the captured, processed and evaluated retinal image to a cloud server for extensive processing. Further, the processing of the retinal image facilitates determining 6 eye related diseases, and deriving abnormalities at the heart, brain and kidneys.

These and other aspects of the embodiments herein will be better appreciated and understood when considered in conjunction with the following description and the accompanying drawings. It should be understood, however, that the following descriptions, while indicating the preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates an arrangement of for a system for an Intelligent Single Tool for Clinicians for detecting abnormalities related to Opthalmology, Cardiology, Neurology, and Nephrology of a patient, according to an embodiment of the present invention.

FIG. 2 illustrates a block diagram of for an Intelligent Single Tool for Clinicians for detection and derivation of eye diseases, and ailments at brain, kidney and heart, according to an embodiment of the present invention.

FIG. 3 illustrates the details of performance, measure of segmentation process of an Intelligent Single Tool for Clinicians, according to an embodiment of the present invention.

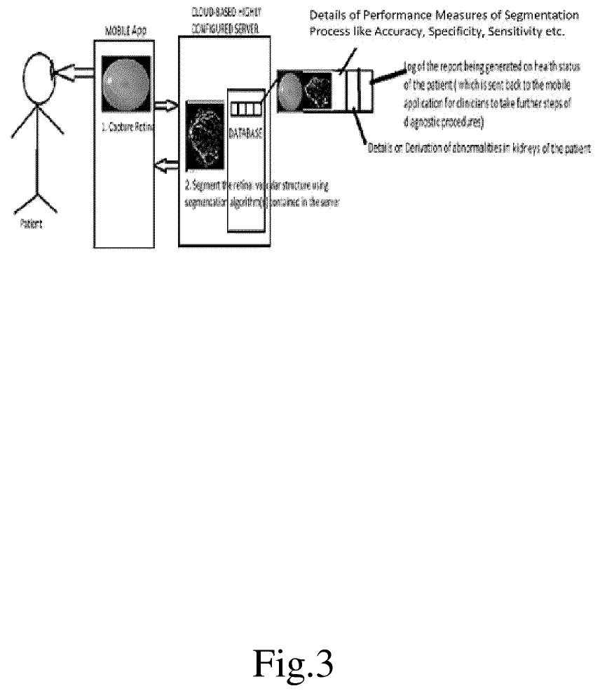

Although the specific features of the present invention are shown in some drawings and not in others. This is done for convenience only as each feature may be combined with any or all of the other features in accordance with the present invention.

### DETAILED DESCRIPTION OF THE INVENTION

In the following detailed description, a reference is made to the accompanying drawings that form a part hereof, and in which the specific embodiments that may be practiced is shown by way of illustration. These embodiments are described in sufficient detail to enable those skilled in the art to practice the embodiments and it is to be understood that other changes may be made without departing from the scope of the embodiments. The following detailed description is therefore not to be taken in a limiting sense. The various embodiments of the present invention for a system for an Intelligent Single Tool for Clinicians for detecting abnormalities in Opthalmology, Cardiology, Neurology, and Nephrology of a patient.

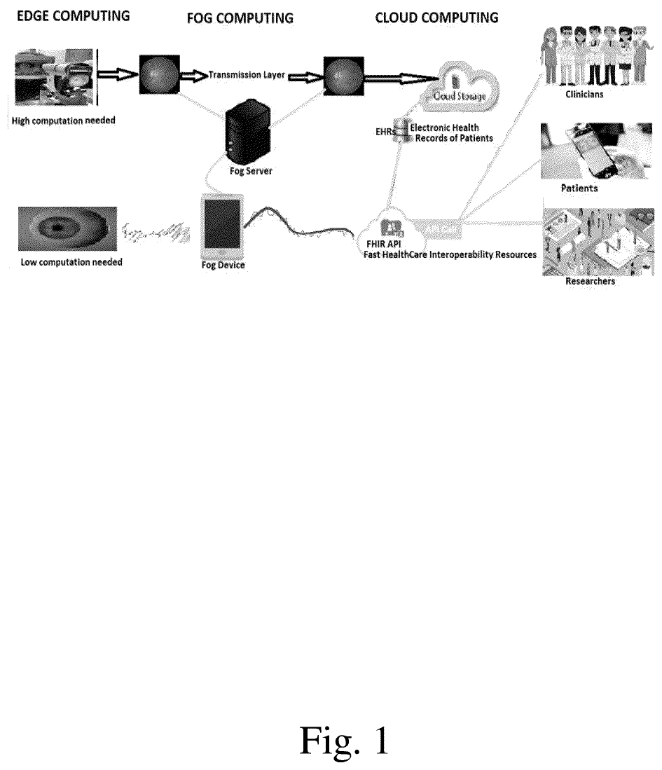

As illustrated in FIG. 1 The various embodiments of the present invention provide a system for an Intelligent Single Tool for Clinicians for detecting abnormalities in Opthalmology, Cardiology, Neurology, and Nephrology of a patient, the system comprises an smart device with an TOT sensor and an external server, wherein the IOT sensor is an edge device with a communication unit, wherein the IOT sensor capture a retinal image of the patient to process the image, wherein to determine one or mom eye related diseases; and a plurality of other diseases relaxed to one or more of cardio-vascular, renal failure, nephropathy, and kidney.

According to an embodiment FIG. 1 the IOT sensor capture the retinal image through Handheld Fundus Camera (HF-Camera). The said camera is either connected to a standalone smart device or captured through smart phone.

According to an embodiment of the present invention, the system further comprising a communication unit to receive electronic health records (EHR) via a FHIR (Fast Healthcare Interoperability Resources) APT (application programming interface) in the smart device.

According to an embodiment the processing of image comprising of two steps, a Pre-Processing step and a Post-Processing step, the Pre-Processing step consist of (a) Optimization of the image wherein to avoid working with complete set of pixels of the captured input image, optimization of the input image, (b) Conversion of input RGB image to gray image, is an important step in image processing while conversion of an input RGB image to gray, Feature Extraction is done parallel with Vector-Calculus techniques including Principal Component Analysis, Singular Value Decomposition, Independent Component Analysis etc. and (c) Smoothing wherein the noise is removed by using filters like Gaussian Filter, increasing contrast using Contrast Limited Adaptive Histogram Equalisation of the image, for making the image to be processed effectively to generate mom accurate segmentation of the retinal image.

Furthermore the Post-Processing may span one or at least two set of steps consist of (a) Classifying or Clustering the set of retinal blood vessel pixels using the most effective tools like Fuzzy C-Means Clustering/Support Vector Machines/K-Means Clustering/Maximum Principal Curvatures in combination with Structuring Elements of MATLAB/Thresholding Techniques of Image Processing etc. As on the required, integration of Deep Learning techniques in bringing accurate segmentaion will be employed. (b) level Set/Active Contours for perfect evolution of Blood-Vessel Structure to perfectly lock the segmented image for elegant evaluation. (c) Assessment of the segmented image is done through performance measures including Accuracy. Specificity, Sensitivity etc to validate the procedure opted to get segmented image is successful or not.

According to an embodiment of the present invention for diagnosis the communication unit to send data related to the processed image to a cloud server for analyzing long-term patterns of the patient, after determining whether the processing greater than processing capability of edge device is required.

According to an embodiment of the present invention the IOT sensor is an inertial measurement unit (IMU)-based actigraph configured to send data real time to a fog device that contains higher computing power that an edge device for gesture recognition.

According to an embodiment of the present invention the IOT sensor to clean data related to the retinal image and facilitate the fog node to handle sensor fusion of clinical one-dimensional (1D) biosignal.

According to an embodiment of the present invention the system, wherein a fog device is used to process the image, when the processing required is higher than the processing capability of the edge device.

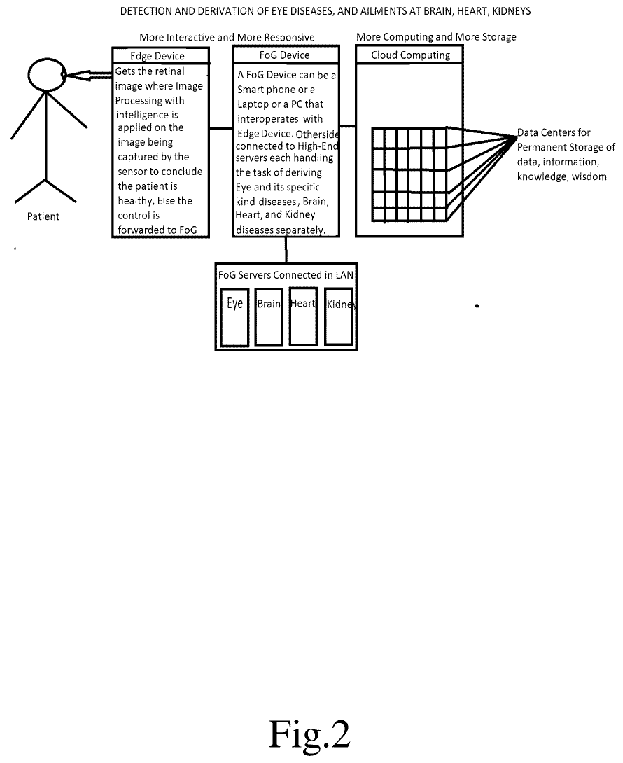

According to an embodiment as illustrated in FIG. 2 after completing processing the image, the external server or the cloud server compares the processed image as a record in the database table for future reference. Here, the database table will get populated with the following set of details (a) Initial image (input) captured by mobile application or by FOG device (if required is sent to the cloud-based server for pre and post processing). (b) Segmented image (output) being generated by the in-house techniques available with the server. The pre-processing and post-processing techniques discussed above will be applied to get a segmented image with higher accuracy. (c) Details on abnormalities at kidneys which are derived from the segmented image. (d) Report generated on the health status of the patient (this health status is sent back to the mobile application), based on which the clinicians proceed with the further diagnostic procedures.

According to an embodiment of the present invention A method for detecting abnormalities in brain, heart and kidney of a patient, the method comprising a method for capturing the retinal image by an TOT sensor, further processing the retinal image using one of the IOT sensor and a FOG device, wherein the IOT sensor is an edge device and the FOG device consists of a mobile application; and sending the captured retinal image to a cloud server for extensive processing. Further the processing of the retinal image facilitates determining 6 eye related diseases, and deriving abnormalities at the heart, brain and kidneys.

The foregoing description of the specific embodiments will so fully reveal the general nature of the embodiments herein that others can, by applying current knowledge, readily modify and/or adapt for various applications such as specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modifications. However, all such modifications are deemed to be within the scope of the claims.

I/We claim:

1. A system for an Intelligent Single Tool for Clinicians for detecting abnormalities in Opthalmology, Cardiology, Neurology, and Nephrology of a patient, the system comprises:
a smart device with an IOT sensor to:
capture a retinal image of the patient;
process the image;
determine one or more eye related diseases and a plurality of other diseases related to one or more of cardio-vascular, renal failure, nephropathy, and kidney, wherein the IOT sensor is an edge device; and
a communication unit to send data related to the processed mage to a cloud server for analyzing long-term patterns of the patient, when processing greater than processing capability of edge device is required.

2. The system of an Intelligent Single Tool for Clinicians as claimed in claim 1, wherein the IOT sensor is an inertial measurement unit (IMU)-based actigraph configured to send data real time to a fog device that contains higher computing power that an edge device for gesture recognition.

3. The system of an Intelligent Single Tool for Clinicians as claimed in claim 1, further comprising:
a communication unit to receive electronic health records (EHR) via a FHIR (Fast Healthcare Interoperability Resources) API (Application Programming Interface).

4. The system of an Intelligent Single Tool for Clinicians as claimed in claim 1, wherein the IOT sensor to clean data related to the retinal image and facilitate the fog node to handle sensor fusion of clinical one-dimensional (1D) biosignal.

5. The system of an Intelligent Single Tool for Clinicians as claimed in claim 1, wherein a fog device is used to process the image, when the processing required is higher than the processing capability of the edge device.

6. A method for an Intelligent Single Tool for Clinicians for detecting abnormalities in eyes, brain, heart and kidney of a patient, the method comprising:
capturing the retinal image by an IOT sensor;
processing the retinal image using one of the IOT sensor and a FOG device, wherein the IOT sensor is an edge device and the FOG device consists of a mobile application; and
sending the captured retinal image to a cloud server for extensive processing.

7. The method of an Intelligent Single Tool for Clinicians as claimed in claim 6, wherein the processing of the retinal image facilitates determining 6 eye related diseases, and deriving abnormalities at the heart, brain and kidneys.
